(19) United States
(12) Patent Application Publication
(10) Pub. No.: US 2007/0293514 A1
(43) Pub. Date: Dec. 20, 2007

(54) Triazolopyrimidines

(75) Inventors: Olaf Gebauer, Leverkusen (DE); Herbert Gayer, Monheim (DE); Ulrich Heinemann, Leichlingen (DE); Stefan Herrmann, Langenfeld (DE); Stefan Hillebrand, Neuss (DE); Hans-Ludwig Elbe, Wuppertal (DE); Ronald Ebbert, Nurnberg (DE); Ulrike Wachendorff-Neumann, Neuwied (DE); Peter Dahmen, Neuss (DE); Karl-Heinz Kuck, Langenfeld (DE)

Correspondence Address: BAYER CROPSCIENCE LP, Patent Department, 2 T .W. ALEXANDER DRIVE, RESEARCH TRIANGLE PARK, NC, 27709, US

(73) Assignee: BAYER CROPSCIEMCE AG, 40789 MONHEIM GERMANY (DE)

(21) Appl. No.: 10/583,836

(22) PCT Filed: Dec. 22, 2004
(86) PCT No.: PCT/EP04/14592

§ 371 Date: Mar. 26, 2007

(30) Foreign Application Priority Data
Dec. 22, 2003 (DE) 10360370.0

(51) Int. Cl. A01N 43/90; A01N 55/10; A01P 3/00; A01P 7/04; C07D 487/04; C07F 7/10

(52) U.S. Cl. 514/262.1; 514/63; 544/229; 544/263

(57) ABSTRACT

This invention relates to triazolopyrimidines-of the formula 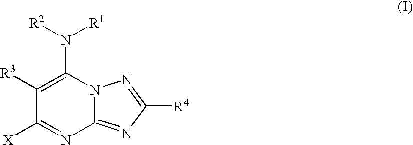
in which R¹, R², R³, R⁴ and X are as defined in the disclosure, to a process for preparing these compounds and to their use for controlling unwanted microorganisms.

The invention relates to triazolopyrimidines, to a process for their preparation and to their use for controlling unwanted microorganisms.

It is already known that certain triazolopyrimidines have fungicidal properties (cf. EP 0 550 113-A, WO 94-20501, EP 0 613 900-A, U.S. Pat. No. 5,612,345-A, EP 0 834 513-A, WO 98-46 607 and WO 98-46 608). The activity of these compounds is good; however, at low application rates it is sometimes unsatisfactory.

However, since the ecological and economical demands made on modern fungicides are increasing constantly, for example with respect to activity spectrum, toxicity, selectivity, application rate, formation of residues and favorable manufacture, and there can furthermore be problems, for example, with resistance, there is a constant need to develop novel fungicides which, at least in some areas, have advantages over those of the prior art.

This invention now provides novel triazolopyrimidines of the formula 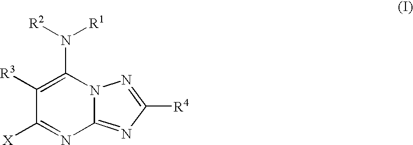
in which
R¹ represents H, R², optionally substituted alkyl, optionally substituted alkenyl, optionally substituted alkynyl, optionally substituted cyloalkyl or represents optionally substituted heterocyclyl;
R² represents an organic radical which contains 3 to 13 carbon atoms and one or more silicon atoms and, if appropriate, 1 to 3 identical or different heteroatoms from the group consisting of oxygen, nitrogen and sulfur, and which is unsubstituted or substituted by 1 to 4 identical or different halogens; or
R¹ and R² together with the nitrogen atom to which they are attached represent an optionally substituted heterocyclic ring which contains one or more silicon atoms and/or is substituted by one or more radicals R²;
R³ represents optionally substituted aryl, optionally substituted heterocyclyl, optionally substituted alkyl, optionally substituted alkenyl, optionally substituted alkynyl, optionally substituted cycloalkyl, optionally substituted aralkyl, or optionally substituted amino group, optionally substituted (C₁-C₈)-alkoxy, optionally substituted (C₁-C₈)-alkylthio, optionally substituted (C₆-C₁₀)-aryloxy, optionally substituted (C₆-C₁₀)-arylthio, optionally substituted heterocyclyloxy, optionally substituted heterocyclyloxy, optionally substituted C₆-C₁₀)-aryl-(C₁-C₄)-alkoxy, optionally substituted (C₆-C₁₀)-aryl-(C₁-C₄)-alkylthio, optionally substituted heterocyclyl-(C₁-C₄)-alkoxy, or optionally substituted heterocyclyl-(C₁-C₄)-alkylthio;
R⁴ represents H, halogen, optionally halogen-substituted alkyl or optionally halogen-substituted cycloalkyl and
X represents halogen, cyano, optionally substituted alkyl, optionally substituted alkoxy, optionally substituted alkylthio, optionally substituted alkylsulfinyl, optionally substituted alkylsulfonyl or optionally substituted phenyl,
   and their salts.

Furthermore, it has been found that triazolopyrimidines of the formula (I) can be prepared by
(a) reacting halotriazolopyrimidines of the formula 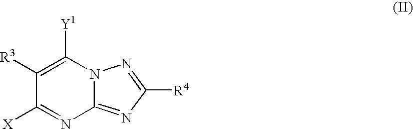
in which
R³, R⁴ and X are as defined above and
Y¹ represents halogen,
with amines of the formula 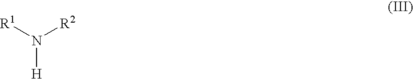
in which
R¹ and R² are as defined above,
if appropriate in the presence of a diluent, if appropriate in the presence of an acid acceptor and if appropriate in the presence of a catalyst.

Finally, it has been found that the triazolopyrimidines of the formula (I) are highly suitable for controlling unwanted microorganisms. Especially, they have strong fungicidal activity and can be used both in crop protection and in the protection of materials.

The compounds according to the invention of formula (I) can, if appropriate, be present as mixtures of different possible isomeric forms, in particular stereoisomers, such as E and Z, threo and erythro, and also optical isomers, such as R and S isomers or atrope isomers, and, if appropriate, also of tautomers.

The formula (I) provides a general definition of the triazolopyrimidines according to the invention.

Preference is given to compounds of the formula (I) in which

a¹) R³ represents optionally substituted aryl, or

a²) R³ represents optionally substituted heterocyclyl, or

a³) R³ represents optionally substituted alkyl, or

a⁴) R³ represents optionally substituted alkenyl, or

a⁵) R³ represents optionally substituted alkynyl, or

a⁶) R³ represents optionally substituted cycloalkyl, or

a⁷) R³ represents optionally substituted aralkyl, or

a⁸) R³ represents an optionally substituted amino group.

Preference is likewise given to compounds of the formula (I) which R³ has one of the meanings below:

b¹: a¹, a², a³, a⁴, a⁵, a⁶, a⁷,

b²: a¹, a², a³, a⁴, a⁵, a⁶, a⁸,

b³: a¹, a², a³, a⁴, a⁵, a⁷, a⁸,

b⁴: a¹, a², a³, a⁴, a⁶, a⁷, a⁸,

b⁵: a¹, a², a³, a⁵, a⁶, a⁷, a⁸,

b⁶: a¹, a², a⁴, a⁵, a⁶, a⁷, a⁸,

b⁷: a¹, a³, a⁴, a⁵, a⁶, a⁷, a⁸,

b⁸: a², a³, a⁴, a⁵, a⁶, a⁷, a⁸.

Preference is furthermore given to those compounds of the formula (I) in which one or more symbols have one of the preferred meanings given below, i.e. in which
R¹ represents H, or
R¹ represents a radical R², or
R¹ represents alkyl having 1 to 6 carbon atoms which may be mono- to pentasubstituted by identical or different substituents from the group consisting of halogen, cyano, hydroxy, alkoxy having 1 to 4 carbon atoms and cycloalkyl having 3 to 8 carbon atoms, or
R¹ represents alkenyl having 2 to 6 carbon atoms which may be mono- to trisubstituted by identical or different substituents from the group consisting of halogen, cyano, hydroxy, alkoxy having 1 to 4 carbon atoms and cycloalkyl having 3 to 8 carbon atoms, or
R¹ represents alkynyl having 3 to 6 carbon atoms which may be mono- to trisubstituted by identical or different substituents from the group consisting of halogen, cyano, alkoxy having 1 to 4 carbon atoms and cycloalkyl having 3 to 8 carbon atoms, or
R¹ represents cycloalkyl having 3 to 8 carbon atoms which may be mono- to trisubstituted by identical or different substituents from the group consisting of halogen and alkyl having 1 to 4 carbon atoms, or
R¹ represents saturated or unsaturated heterocyclyl having 3 to 8 ring members and 1 to 3 heteroatoms, such as nitrogen, oxygen and/or sulfur, where the heterocyclyl may be mono- or disubstituted by halogen, alkyl having 1 to 4 carbon atoms, cyano and/or cycloalkyl having 3 to 8 carbon atoms,
R² represents an aliphatic saturated or unsaturated group having 1 to 13 carbon atoms and one or more silicon atoms which optionally contains 1 to 3 identical or different heteroatoms from the group consisting of oxygen, sulfur and nitrogen and which is unsubstituted or substituted by 1 to 4 identical or different halogen atoms, or
R¹ and R² together with the nitrogen atom to which they are attached represent a saturated or unsaturated heterocyclic ring having 3 to 8 ring members which contains one or more silicon atoms and/or is substituted by one or more radicals R², where the heterocycle may contain a further nitrogen, oxygen or sulfur atom as ring member and where the heterocycle may furthermore be substituted up to three times by fluorine, chlorine, bromine, alkyl having 1 to 4 carbon atoms and/or haloalkyl having 1 to 4 carbon atoms and 1 to 9 fluorine and/or chlorine atoms,
R³ represents C₁-C₁₀-alkyl, C₂-C₁₀-alkenyl, C₂-C₁₀-alkynyl, C₃-C₈-cycloalkyl, phenyl-C₁-C₁₀-alkyl where R³ is unsubstituted or partly or fully halogenated and/or optionally carries one to three radicals from the group R^{x}, or C₁-C₁₀-halogenalkyl which optionally carries one to three radicals from the group R^{x}, and R^{x} represents cyano, nitro, hydroxy, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-halogenalkylthio, C₁-C₆-alkylsulfinyl, C₁-C₆-halogenalkylsulfinyl, C₁-C₆-alkylsulfonyl, C₁-C₆-halogenalkylsulfonyl, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and optionally halogenated oxy-C₁-C₄-alkyl-C₁-C₄-alkeneoxy, oxy-C₁-C₄-alkenyl-C₁-C₄-alkoxy, oxy-C₁-C₄-alkyl-C₁-C₄-alkyloxy,
R³ represents phenyl which may be mono- to tetrasubstituted by identical or different substituents from the group consisting of
   halogen, cyano, nitro, amino, hydroxy, formyl, carboxy, carbamoyl, thiocarbamoyl;
   in each case straight-chain or branched alkyl, alkoxy, alkylthio, alkylsulfinyl or alkylsulfonyl having in each case 1 to 6 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkylsulfinyl or haloalkylsulfonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 11 identical or different halogen atoms;
   in each case straight-chain or branched alkylamino, dialkylamino, alkylcarbonyl, alkylcarbonyloxy, alkoxycarbonyl, alkylsulfonyloxy, hydroximinoalkyl or alkoximinoalkyl having in each case 1 to 6 carbon atoms in the individual alkyl moieties; cycloalkyl having 3 to 8 carbon atoms;
   2,3-attached 1,3-propanediyl, 1,4-butanediyl, methylenedioxy (—O—CH₂—) or 1,2-ethylenedioxy (—O—CH₂—CH₂—O—), where these radicals may be mono- or polysubstituted by identical or different substituents from the group consisting of halogen, alkyl having 1 to 4 carbon atoms and haloalkyl having 1 to 4 carbon atoms and 1 to 9 identical or different halogen atoms;
      or
R³ represents saturated or unsaturated heterocyclyl having 3 to 8 ring members and 1 to 3 heteroatoms from the group consisting of nitrogen, oxygen and sulfur, where the heterocyclyl may be mono- or disubstituted by halogen, alkyl having 1 to 4 carbon atoms, alkoxy having 1 to 4 carbon atoms, alkylthio having 1 to 4 carbon atoms, haloalkoxy having 1 to 4 carbon atoms, haloalkylthio having 1 to 4 carbon atoms, cyano, nitro and/or cycloalkyl having 3 to 6 carbon atoms;
   or
R³ represents C₁-C₈-alkylamino, C₂-C₈-alkenylamino, C₂-C₈-alkynylamino, di-C₁-C₈-alkylamino, di-C₂-C₈-alkenylamino, di-C₂-C₈-alkynylamino, C₂-C₈-alkenyl-(C₂-C₈)-alkynylamino, C₂-C₆-alkynyl-(C₁-C₈)-alkylamino, C₂-C₈-alkenyl-(C₁-C₈)-alkylamino, C₆-C₁₀-arylamino, C₆-C₁₀-aryl-(C₁-C₈)-alkylamino, C₆-C₁₀-aryl-(C₁-C₄)-alkyl-(C₁-C₈)-alkylamino, heterocyclyl-(C₁-C₈)-alkylamino or heterocyclyl-(C₁-C₄)-alkyl-(C₁-C₈)-alkylamino;
R⁴ represents H, halogen, (C₁-C₄)-alkyl which is unsubstituted or substituted by one or more halogen atoms, cyclopropyl which is unsubstituted or substituted by one or more halogen atoms, and
X represents fluorine, chlorine, bromine, CN, (C₁-C₄)-alkyl which is unsubstituted or substituted by one or more fluorine or chlorine atoms, (C₁-C₄)-alkoxy which is unsubstituted or substituted by one or more fluorine or chlorine atoms or (C₁-C₄)-alkylthio which is unsubstituted or substituted by one or more fluorine or chlorine atoms.

Particular preference is given to those triazolopyrimidines of the formula (I) in which one or more symbols have one of the particularly preferred meanings listed below, i.e. in which
R¹ represents hydrogen, methyl or ethyl, or
R² represents a group of the formula Y²—Si(OₘCH₃)(OₙCH₃)(OₚY³),
   where m, n and p independently of one another represent 0 or 1;
   Y² represents a bond or alkanediyl, alkenediyl or alkynediyl, each of which is straight-chain or branched, has 1 to 6 or 2 to 6 carbon atoms, is optionally interrupted by one or two nonadjacent oxygen atoms and is unsubstituted or substituted by one to three identical or different halogen atoms;
   Y³ represents straight-chain or branched alkyl or alkenyl having 1 to 5 or 2 to 5 carbon atoms, optionally interrupted by an oxygen-nitrogen or sulfur atom and unsubstituted or substituted by 1 to 3 identical or different halogen atoms;
R³ represents (C₁-C₈)-alkyl, (C₁-C₈)-cycloalkyl or benzyl or
R³ represents phenyl which may be mono- to trisubstituted by identical or different substituents from the group consisting of
   fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl or cyclohexyl,
   2,3-attached 1,3-propanediyl, 1,4-butanediyl, methylenedioxy (—O—CH₂—O—) or 1,2-ethylenedioxy (—O—CH₂—CH₂—O—), where these radicals may be mono- or polysubstituted by identical or different substituents from the group consisting of fluorine, chlorine, methyl, ethyl, n-propyl, i-propyl and trifluoromethyl.
R³ represents pyridyl which is attached in the 2- or 4-position and may be mono- to tetrasubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl, or
R³ represents pyrimidyl which is attached in the 2- or 4-position and may be mono- to trisubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl, or
R³ represents thienyl which is attached in the 2- or 3-position and may be mono- to trisubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl, or
R³ represents C₁-C₈-alkylamino or di-C₁-C₈-alkylamino, or
R³ represents thiazolyl which is attached in the 2-, 4- or 5-position and may be mono- to trisubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl, or
R³ represents N-piperidinyl, N-tetrazolyl, N-pyrazolyl, N-imidazolyl, N-1,2,4-triazolyl, N-pyrrolyl, or N-morpholinyl, each of which is unsubstituted or mono- or—if possible—polysubstituted by identical or different substituents from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl,
R⁴ represents H, Cl, F, CH₃, —CH(CH₃)₂ or cyclopropyl; and
X represents F, Cl, CN, (C₁-C₄)-alkyl which is unsubstituted or substituted by one or more fluorine or chlorine atoms, OCH₃ or SCH₃.

Very particular preference is given to compounds of the formula (I) in which one or more of the symbols have one of the very particularly preferred meanings listed below, in which
R¹ represents H;
R² represents SiMe₃, SiMe₂Et, SiMe₂CHMe₂, SiMe₂CH₂CHMe₂, SiMe₂CH₂CMe₃, SiMe₂OCHMe₂, SiMe₂OCH₂CHMe₂, CH₂SiMe₃, CH₂SiMe₂Et, CH₂SiMe₂CHMe₂, CH₂SiMe₂CH₂CHMe, CH₂SiMe₂OMe, CH₂SiMe₂OCHMe₂, CH₂SiMe₂OCH₂CHMe₂, CHMeSiMe₃, CHMeSiMe₂OMe, (CH₂)₂SiMe₃, (CH₂)₂SiMe₂Et, (CH₂)₂SiMe₂CHMe₂, (CH₂)₂SiMe₂CMe₃, (CH₂)₂SiMe₂CH₂CHMe₂, (CH₂)₂SiMe₂CH₂CH₂Me, (CH₂)₂SiMe₂CH₂CMe₃, (CH₂)₂SiMe₂OCHMe₂, (CH₂)₂SiMe₂OCH₂CHMe₂, CHMeCH₂SiMe₃, CHMeCH₂SiMe₂Et, CHMeCH₂SiMe₂CH₂CH₂Me, CHMeCH₂SiMe₂CHMe₂, CHMeCH₂SiMe₂CMe₃, CHMeCH₂SiMe₂CH₂CHMe₂, CFMeCH₂SiMe₃, CHMeCH₂CH₂SiMe₂OMe, CHMeCH₂SiMe₂OCHMe₂, CHMeCH₂SiMe₂OCH₂CHMe₂, CH₂CHMeSiMe₃, CH₂CHMeSiMe₂Et, CH₂CHMeSiMe₂CHMe₂, CHMeCHMeSiMe₃, CMe₂CH₂SiMe₃, (CH₂)₃SiMe₃, (CH₂)₃SiMe₂Et, (CH₂)₃SiMe₂CHMe₂, (CH₂)₃SiMe₂CH₂CHMe₂, (CH₂)₃SiMe₂OMe, (CH₂)₃SiMe₂OCHMe₂, (CH₂)₃SiMe₂OCH₂CHMe₂, CHMeCH₂CH₂SiMe₃, CHMeCH₂CH₂SiMe₂Et, CHMeCH₂CH₂SiMe₂CHMe₂, CHMeCH₂CH₂CH₂SiMe₂OMe, CHMeCH₂CH₂SiMe₂OCHMe₂, CMe=CHSiMe₃, CH₂CH₂SiMe₂OMe, —C≡C—SiMe₃, —CH₂—C≡C—SiMe₃ or —CHMe-C≡C—SiMe₃;
R³ represents (C₁-C₆)-alkyl, (C₃₋₆)-alkenyl, (C₃-C₆)-alkynyl, (C₃-C₈)-cycloalkyl, where R³ is unsubstituted or substituted by one or more fluorine or chlorine atoms,
   or
R³ represents 2,4- or 2,6-disubstituted phenyl, or represents 2-substituted phenyl or represents 2,4,6-trisubstituted phenyl,
R³ represents pyridyl which is attached in the 2- or 4-position and may be mono- to tetrasubstituted by identical or different substituents from the group consisting of fluorine, chlorine, bromine, cyano, methyl, ethyl, methoxy, methylthio, hydroximinomethyl, hydroximinoethyl, methoximinomethyl, methoximinoethyl and trifluoromethyl, or
R³ represents pyrimidyl which is attached in the 4-position and may be mono- to trisubstituted by identical or different substituents from the group consisting of fluorine, chlorine, bromine, cyano, methyl, ethyl, methoxy, methylthio, hydroximinomethyl, hydroximinoethyl, methoximinomethyl, methoximinoethyl and trifluoromethyl;
R⁴ represents H, —CH₃, —CH(CH₃)₂, Cl or cyclopropyl,
   and
X represents fluorine, chlorine, CN, (C₁-C₃)-alkyl, in particular CH₃ or (C₁-C₃)-haloalkyl, in particular CF₃, OCH₃, or SCH₃.

The radical definitions mentioned above can be combined with one another as desired. Moreover, individual definitions may not apply.

Using, for example, 5,7-dichloro-6-(5-chloropyrimidin-4-yl)-[1,2,4]triazolo[1,5-a]pyrimidine as starting material, the course of the process (a) according to the invention can be illustrated by the formula scheme below.

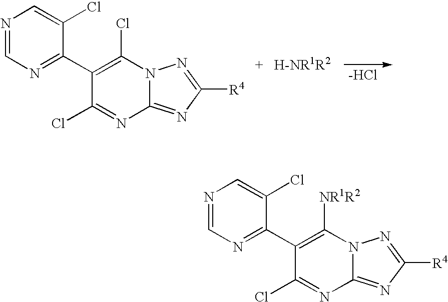

The formula (II) provides a general definition of the dihalotriazolopyrimidines required as starting materials for carrying out the process (a) according to the invention. In this formula (II), R³ and X preferably have those meanings which have already been mentioned in connection with the description of the compounds of the formula (I) according to the invention as being preferred for these radicals. Y¹ preferably represents fluorine, chlorine or bromine, particularly preferably fluorine or chlorine.

Dihalotriazolopyrimidines can be prepared by reacting, for example,
(b) dihydroxytriazolopyrimidines of the formula 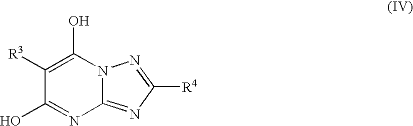
in which
R³ is as defined above,
with halogenating agents, if appropriate in the presence of a diluent.

Using, for example, 6-(5-chloropyrimidin-4-yl)-[1,2,4]triazolo[1,5-a]-pyrimidine-5,7-diol as starting material and phosphorus oxychloride in a mixture with phosphorus pentachloride as halogenating agent, the course of the process (b) according to the invention can be illustrated by the formula scheme below.

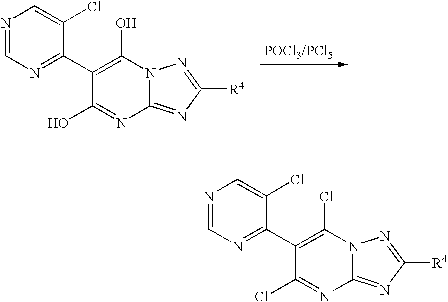

The formula (IV) provides a general definition of the dihydroxytriazolopyrimidines required as starting materials for carrying out the process (b). In this formula, R³ preferably has those meanings which have already been mentioned in connection with the description of the compounds of the formula (I) according to the invention as being preferred for this radical.

Dihydroxytriazolopyrimidines of the formula (IV) can be prepared by reacting, for example,
(c) heteroarylmalonic esters of the formula 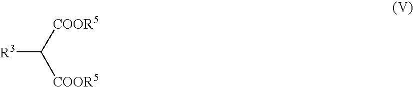
in which
R³ is as defined above and
R⁵ represents alkyl having 1 to 4 carbon atoms,
with an aminotriazole of the formula 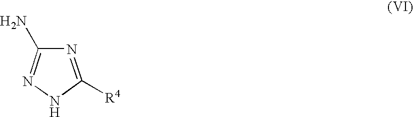
if appropriate in the presence of a diluent and if appropriate in the presence of an acid binder.

Using, for example, dimethyl 2-(5-chloropyrimidin-4-yl)malonate and a 3-aminotriazole as starting materials, the course of the process (c) according to the invention can be illustrated by the formula scheme below.

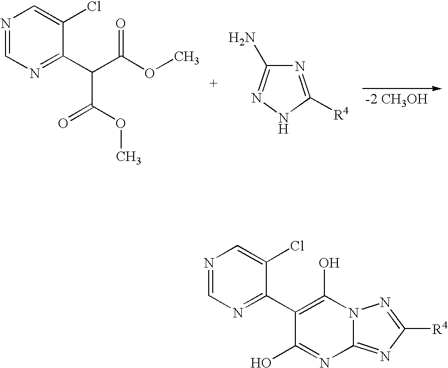

The formula (V) provides a general definition of the heteroarylmalonic esters required as starting materials for carrying out the process (c) according to the invention. In this formula, R³ preferably has those meanings which have already been mentioned in connection with the description of the compounds of the formula (I) according to the invention as being preferred for this radical. R⁵ represents methyl or ethyl.

The heteroarylmalonic esters of the formula (V) are known (cf. DE 38 20 538-A, WO 01-11 965 and DE-A 103 25 133).

Using, for example, 2-chloro-3-trifluoromethylpyridine and dimethyl malonate as starting materials, the course of the process (d) according to the invention can be illustrated by the formula scheme below.

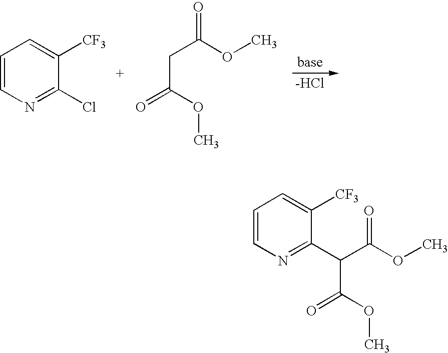

The halopyridines required as starting materials for carrying out the process (d) according to the invention are known chemicals for synthesis.

The malonic esters furthermore required as starting materials for carrying out the process (d) according to the invention are likewise known chemicals for synthesis.

Using, for example, 4,5-dichloropyrimidine and dimethyl malonate as starting materials, the course of the process (e) according to the invention can be illustrated by the formula scheme below.

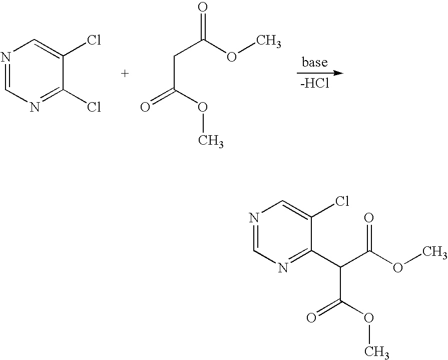

The halopyrimidines required as starting materials for carrying out the process (e) according to the invention are known and can be prepared with known methods (cf. J. Chem. Soc. 1955, 3478, 3481).

The aminotriazoles of the formula (VI) furthermore required as starting material for carrying out the process (c) according to the invention are commercial chemicals.

Suitable halogenating agents for carrying out the process (b) are all components customary for replacing hydroxyl groups by halogen. Preference is given to using phosphorus trichloride, phosphorus tribromide, phosphorus pentachloride, phosphorus oxychloride, thionyl chloride, thionyl bromide, phosgene, diphosgene, triphosgene or mixtures thereof, in particular a mixture of phosphorus oxychloride, phosphorus trichloride and chlorine (see, for example, EP-A 1 077 210). The corresponding fluorine compounds of the formula (II) can be prepared from the chlorine or bromine compounds by reaction with potassium fluoride.

The halogenating agents mentioned are known.

The formula (III) provides a general definition of the amines required as starting materials for carrying out the process (a) according to the invention. In this formula, R¹ and R² preferably have those meanings which have already been mentioned in connection with the description of the compounds of the formula (I) according to the invention as being preferred for R¹ and R².

The synthesis of compounds of the formula (II) in which R³ represents an alkyl, alkenyl, alkynyl or cycloalkyl group is described in WO-A 03/009687.

The synthesis of compounds of the formula (II) in which R³ carries an amino, alcohol or thio function is described in WO-A 03/039259.

The amines of the formula (III) are known. Some of them are commercially available, or they can be prepared by known methods familiar to the person skilled in the art.

Thus, silylated amines of the formula (IIIa)
H₂N—(CR^{a}R^{b})ₙ—SiR^{c}R^{d}R^{e}
in which
n is a natural number from 0 to 10 and
R^{a}, R^{b}, R^{c}, R^{d} are identical or different and H, CH₃ or C₂H₅ (the total number of carbon atoms in R^{a-d} being ≦12),
   are generally obtainable, for example by reacting phthalimide in the presence of a base, such as K₂CO₃, with a haloalkylsilane and cleaving the resulting N-substituted phthalimide with hydrazine:

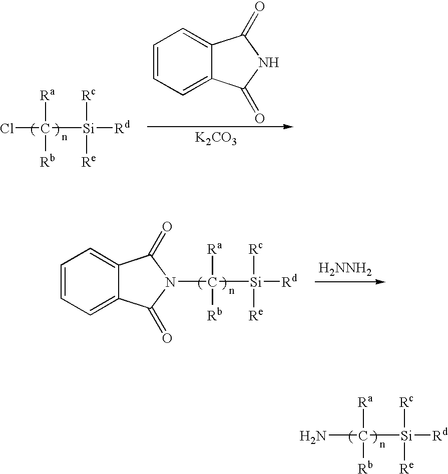

Such syntheses are described, for example, in J. Am. Chem. Soc. 1951, 73, 5130 or J. Organomet. Chem. 1978, 174, C18.

Haloalkylsilanes are commercially available or can be prepared by known methods familiar to the person skilled in the art (see, for example, Houben-Weyl, volume 13/5, p. 65 ff. or Science of Synthesis, vol. 4, p. 247 ff.).

Suitable diluents for carrying out the process (a) according to the invention are all customary inert organic solvents. Preference is given to using halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulfoxides, such as dimethyl sulfoxide; sulfones, such as sulfolane.

Suitable acid acceptors for carrying out the process (a) according to the invention are all inorganic or organic bases customary for such reactions. Preference is given to using alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, and furthermore ammonium compounds such as ammonium hydroxide, ammonium acetate and ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable catalysts for carrying out the process (a) according to the invention are all reaction promoters customary for such reactions. Preference is given to using fluorides, such as sodium fluoride, potassium fluoride or ammonium fluoride.

When carrying out the process (a) according to the invention, the reaction temperatures can be varied within a relatively wide range. In general, the process is carried out at temperatures between 0° C. and 150° C., preferably at temperatures between 0° C. and 80° C.

When carrying out the process (a) according to the invention, in general from 0.5 to 10 mol, preferably from 0.8 to 2 mol, of amine of the formula (III) are employed per mole of dihalotriazolopyrimidine of the formula (II). Work-up is carried out by customary methods.

Suitable diluents for carrying out the process (b) according to the invention are all solvents customary for such halogenations. Preference is given to using halogenated aliphatic or aromatic hydrocarbons, such as chlorobenzene. However, it is also possible for the halogenating agent itself, for example phosphorus oxychloride, or a mixture of halogenating agents to act as diluent.

When carrying out the process (b), the temperatures can be also be varied within a relatively wide range. In general, the process is carried out at temperatures between 0° C. and 150° C., preferably between 10° C. and 120° C.

When carrying out the process (b), dihydroxytriazolopyrimidine of the formula (IV) is generally reacted with an excess of halogenating agent. Work-up is carried out by customary methods.

Suitable diluents for carrying out the process (c) are all inert inorganic solvents customary for such reactions. Preference is given to using alcohols, such as methanol, ethanol, n-propanol, i-propanol, n-butanol and tert-butanol.

Suitable acid binders for carrying out the process (c) are all inorganic and organic bases customary for such reactions. Preference is given to using tertiary amines, such as tributylamine or pyridine. It is also possible for excess amine to act as diluent.

When carrying out the process (c), the temperatures can be varied within a relatively wide range. In general, the process is carried out at temperatures between 20° C. and 200° C., preferably between 50° C. and 180° C.

When carrying out the process (c), heteroarylmalonic esters of the formula (V) and aminotriazole of the formula (VI) are generally reacted in equivalent amounts. However, it is also possible to use an excess of one component or the other. Work-up is carried out by customary methods.

Suitable diluents for carrying out the processes (d) and (e) according to the invention are in each case all customary inert organic solvents. Preference is given to using halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; sulfoxides, such as dimethyl sulfoxide; sulfones, such as sulfolane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or else pure water.

Suitable copper salts for carrying out the processes (d) and (e) according to the invention are in each case customary copper salts. Preference is given to using copper(I) chloride or copper(I) bromide.

Suitable acid acceptors for carrying out the processes (d) and (e) according to the invention are in each case all customary inorganic or organic bases. Preference is given to using alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, and furthermore ammonium compounds, such as ammonium hydroxide, ammonium acetate and ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the processes (d) and (e) according to the invention, the reaction temperatures can also be varied within a relatively wide range. In general, the processes are carried out at temperatures between 0° C. and 150° C., preferably at temperatures between 0° C. and 80° C.

When carrying out the process (d) according to the invention, in general from 1 to 15 mol, preferably from 1.3 to 8 mol, of malonic ester are employed per mole of halopyridine. Work-up is carried out by customary methods.

When carrying out the process (e) according to the invention, in general from 1 to 15 mol, preferably from 1.3 to 8 mol, of malonic ester are employed per mole of halopyridine. Work-up is again carried out by customary methods.

The processes according to the invention are generally carried out under atmospheric pressure. However, it is also possible to operate under elevated or reduced pressure.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae; and

Pseudocercosporella species, such as, for example, Pseudocercosporella herpotrichoides.

The active compounds according to the invention also show a strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time for which this protection is achieved generally extends for 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, and of diseases in viticulture and in the cultivation of fruit and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular molds, wood-discoloring and wood-destroying fingi (Basidiomycetes) and against slime organisms and algae.

Microorganisms of the following genera may be mentioned as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulfoxide, or else water. Liquefied gaseous extenders or carriers are to be understood as meaning those liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, corn cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, or else protein hydrolyzates. Suitable dispersants are: for example lignosulfite waste liquors and methylcellulose.

Taclcifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:

2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benalaxyl-M, benodanil; benomyl; benthiavalicarb-isopropyl; benzamacril; benzamacril-isobutyl; bilanafos; binapacryl; biphenyl; bitertanol; blasticidin-S; boscalid; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-A1; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; peftirazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro[4,5]decane-3-amine; sodium tetracarbonate;

and copper salts and preparations, such as Bordeaux mixture; copper hydroxide, copper naphthenate; copper oxychloride; copper sulfate; cufraneb; copper oxide; mancopper; oxine-copper.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/Acaricides/Nematicides:

1. Acetylcholinesterase (AChE) Inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)

1.2 organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorofenvinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorovinphos, thiometon, triazophos, triclorfon, vamidothion)

2. Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))

2.2 oxadiazines (for example indoxacarb)

3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)

3.2 nicotine, bensultap, cartap

4. Acetylcholine Receptor Modulators

4.1 spinosyns (for example spinosad)

5. Antagonists of GABA-Gated Chloride Channels

5.1 cyclodiene organochlorines (for example camphechlor, chlordane, endosulfan, gamma-HCH, HCH, heptachlor, lindane, methoxychlor

5.2 fiproles (for example acetoprole, ethiprole, fipronil, vaniliprole)

6. Chloride Channel Activators

6.1 mectins (for example abamectin, avermectin, emamectin, emamectin-benzoate, ivermectin, milbemectin, milbemycin)

7. Juvenile Hormone Mimetics

(for example diofenolan, epofenonane, fenoxycarb, hydroprene, kinoprene, methoprene, pyriproxifen, triprene)

8. Ecdyson Agonists/Disruptors

8.1 diacylhydrazines (for example chromafenozide, halofenozide, methoxyfenozide, tebufenozide)

9. Chitin Biosynthesis Inhibitors

9.1 benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)

9.2 buprofezin

9.3 cyromazine

10. Inhibitors of Oxidative Phosphorylation, ATP Disruptors

10.1 diafenthiuron

10.2 organotins (for example azocyclotin, cyhexatin, fenbutatin-oxide)

11. Decouplers of Oxidative Phosphorylation Acting by Interrupting the H-Proton Gradient

11.1 pyrroles (for example chlorfenapyr)

11.2 dinitrophenols (for example binapacryl, dinobuton, dinocap, DNOC)

12. Site-I Electron Transport Inhibitors

12.1 METIs (for example fenazaquin, fenpyroximate, pyrimidifen, pyridaben, tebufenpyrad, tolfenpyrad)

12.2 hydramethylnone

12.3 dicofol

13. Site-II Electron Transport Inhibitors

13.1 rotenone

14. Site-III Electron Transport Inhibitors

14.1 acequinocyl, fluacrypyrim

15. Microbial Disruptors of the Insect Gut Membrane

Bacillus thuringiensis strains

16. Inhibitors of Fat Synthesis

16.1 tetronic acids (for example spirodiclofen, spiromesifen)

16.2 tetramic acids [for example 3-(2,5-dimethylphenyl)-8-methoxy-2-oxo-1-azaspiro[4,5]dec-3-en-4-yl ethyl carbonate (alias: carbonic acid, 3-(2,5-dimethylphenyl)-8-methoxy-2-oxo-1-azaspiro[4,5]dec-3-en-4-yl ethyl ester, CAS Reg. No.: 382608-10-8) and carbonic acid, cis-3-(2,5-dimethylphenyl)-8-methoxy-2-oxo-1-azaspiro[4,5]dec-3-en-4-yl ethyl ester (CAS Reg. No.: 203313-25-1)]

17. Carboxamides

(for example flonicamid)

18. Octopaminergic Agonists

(for example amitraz)

19. Inhibitors of Magnesium-Stimulated ATPase

(for example propargite)

20. Phthalamides

(for example N²-[1,1-dimethyl-2-(methylsulfonyl)ethyl]-3-iodo-N′-[2-methyl-4-[1,2,2,2-tetrafluoro-1-(trifluoromethyl)ethyl]phenyl]-1,2-benzenedicarboxamide (CAS Reg. No.: 272451-65-7), flubendiamide)

21. Nereistoxin Analogues

(for example thiocyclam hydrogen oxalate, thiosultap-sodium)

22. Biologicals, Hormones or Pheromones

(for example azadirachtin, Bacillus spec., Beauveria spec., codlemone, Metarrhizium spec., Paecilomyces spec., thuringiensin, Verticillium spec.)

23. Active Compounds with Unknown or Unspecific Mechanisms of Action

23.1 fumigants (for example aluminum phosphide, methyl bromide, sulfinuryl fluoride)

23.2 selective antifeedants (for example cryolite, flonicamid, pymetrozine)

23.3 mite growth inhibitors (for example clofentezine, etoxazole, hexythiazox)

23.4 amidoflumet, benclothiaz, benzoximate, bifenazate, bromopropylate, buprofezin, chinomethionat, chlorodimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,

furthermore the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have a very broad antimycotic activity spectrum in particular against dermatophytes and yeasts, molds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low-volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5 000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soy beans, potatoes, cotton, tobacco, oilseed rape and also fruit plants (with the fruits apples, pears, citrus fruits and grapes), and particular emphasis is given to corn, soy beans, potatoes, cotton, tobacco and oilseed rape. Traits that are particularly emphasized are increased defense of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defense of the plants against fingi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are corn varieties, cotton varieties, soy bean varieties and potato varieties which are sold under the trade names YIELD GARD® (for example corn, cotton, soy beans), KnockOut® (for example corn), StarLink® (for example corn), Boilgard® (cotton), Nucoton® (cotton) and NewLeaf® (potato). Examples of herbicide-tolerant plants which may be mentioned are corn varieties, cotton varieties and soy bean varieties which are sold under the trade names Roundup Ready® (tolerance to glyphosate, for example corn, cotton, soy bean), Liberty Link® (tolerance to phosphinotricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned also include the varieties sold under the name Clearfield® (for example corn). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The compounds of the formula (I) according to the invention are furthermore suitable for suppressing the growth of tumor cells in humans and mammals. This is based on an interaction of the compounds according to the invention with tubulin and microtubuli and by promoting microtubuli polymerization.

For this purpose, it is possible to administer an effective amount of one or more compounds of the formula (I) or pharmaceutically acceptable salts thereof.

The preparation and the use of the active compounds according to the invention is illustrated in the examples below.

### EXAMPLE 1

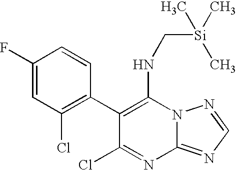

At room temperature, 0.143 g (1.386 mmol) of trimethylsilylmethylamine and 0.216 g (1.890 mmol) of K₂CO₃ were added to a solution of 0.400 g (1.260 mmol) of 5,7-dichloro-6-(2-chloro-4-fluorophenyl)-[1,2,4]triazolo[1,5-a]pyrimidine in 10 ml of acetonitrile, and the mixture was then stirred overnight. The mixture was then stirred into hydrochloric acid, and the precipitated product was washed with water and dried. This gave 0.31 g (58% of theory) of product.

HPLC: logP=3.61

### EXAMPLE 2

### a) Preparation of (1-iodoethyl)trimethylsilane

2.4 g (17.557 mmol) of (1-chloroethyl)trimethylsilane were stirred into 15 ml of acetone, 6.579 g (43.893 mmol) of sodium iodide were added and the mixture was boiled at reflux for 1 day. The mixture was taken up in water and ether and the aqueous phase was extracted twice with ether. The organic phase was dried and the solvent was removed under reduced pressure.

This gave 3.8 g (95% of theory) of product.

### b) (1-Aminoethyl)trimethylsilane

In an autoclave, 3.8 g (16.656 mmol) (1-iodomethyl)trimethylsilane were condensed with 47.670 g (2799.014 mmol) of ammonia, and the mixture was stirred at 135° C. and a pressure of 91 bar for 2.5 hours. The reaction mixture was stirred into CH₂Cl₂, the solvent was removed, 6n aqueous sodium hydroxide solution was added and the mixture was extracted with ether. Removal of the ester under reduced pressure gave 1.4.g (70% of theory) of the product.

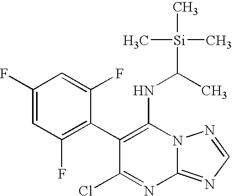

At room temperature, 0.217 g (1.567 mmol) of K₂CO₃ was added to a solution of 0.500 g (1.567 mmol) of 5,7-dichloro-6-(2,4,6-trifluorophenyl)-[1,2,4]triazolo[1,5-a]pyrimidine in 10 ml of acetonitrile and 0.202 g (1.724 mmol) of 1-(trimethylsilyl)ethylamine and the mixture was stirred at room temperature overnight and then at 60° C. for another 6 hours. The solvent was removed under reduced pressure and the residue was taken up in CH₂Cl₃ and 1N hydrochloric acid. Drying and concentration of the organic phase was followed by chromatographic purification (petroleum ether/methyl isobutyl ketone 3:1) of the residue that remained. This gave 0.35 g (51% of theory) of product.

HPLC: logP=3.71

### EXAMPLE 25

a)

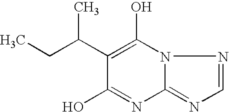

2.6 g (0.012 mol) of diethyl sec-butylmalonate were initially charged in 9.5 g of dimethylformamide. At room temperature, 1 g (0.012 mol) of 3-amino-1,2,4-triazole and 1.8 g (0.013 mol) of 1,8-diazabicyclo(5.4.0)undec-7-ene were added. The mixture was stirred at 100° C. for 6 hours. The mixture was then poured into water and extracted with chloroform. The aqueous phase was concentrated and the residue that remained was stirred with concentrated hydrochloric acid, filtered off with suction and dried.

This gave 0.5 g of 6-sec-butyl-7-hydroxy[1,2,4]triazolo[1,5-a]pyrimidin-5(4H)-one (log p=0.70; HPCL content: 96.2%).
b)

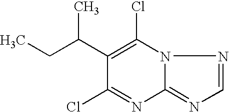

2.6 g (0.093 mol) of diethyl sec-butylmalonate were mixed with 7.8 g (0.093 mol) of 3-amino-1, 2,4-triazole and 19 g (0.102 mol) of tri-n-butylamine, and the mixture was stirred at 180° C. for 6 hours, with distillative removal of the ethanol formed during the reaction. Volatile components were then distilled off at 10 Torr. The residue was reacted further without purification.

The crude 6-sec-butyl-7-hydroxy[1,2,4]triazolo[1,5-a]pyrimidin-5(4H)-one was dissolved in 8.9 g (0.827 mol) of phosphoryl chloride, and 11 g (0.053 mol) of phosphorus pentachloride were added at room temperature. The reaction mixture was heated at 110° C. for 3 hours. Volatile components were then removed at 10 Torr, the residue was taken up in dichloromethane and the solution was washed with ice-water. After drying and distillative removal of the solvent, the residue was chromatographed on silica gel in a mixture of petroleum ether:tert-butyl methyl ether=2:1. This gave 3.5 g of 5,7-dichloro-6-sec-butyl[1,2,4]triazolo[1,5-a]pyrimidine (log p=2.40; HPLC content: 96.4%).
c)

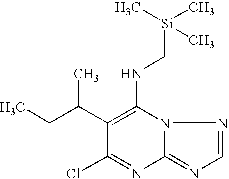

0.5 g (0.002 mol) of 5,7-dichloro-6-sec-butyl[1,2,4]triazolo[1,5-a]pyrimidine was initially charged in 7.8 g of acetonitrile. 0.7 g (0.005 mol) of potassium carbonate and 0.31 g (0.002 mol) of 2-trimethylsilyl-1-aminoethane were added. The reaction mixture was stirred at room temperature for 16 hours. The mixture was then acidified with hydrochloric acid and extracted with diethyl ether. The organic phase was dried and concentrated. The residue was stirred with diethyl ether and then chromatographed on silica gel in a mixture of cychlohexane:ethyl acetate=8:2.

This gave 0.3 g of 5-chloro-6-sec-butyl-7-(1-trimethylsilylethylamino)[1,2,4]triazolo[1,5-a]pyrimidine (log p=3.87; HPLC content: 93%).

### EXAMPLE 26

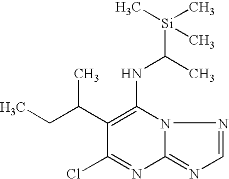

0.5 g (0.002 mol) of 5,7-dichloro-6-sec-butyl[1,2,4]triazolo[1,5-a]pyrimidine was initially charged in 7.8 g of acetonitrile. 0.42 g (0.003 mol) of potassium carbonate and 0.21 g (0.002 mol) of trimethylsilylmethylamine were added. The reaction mixture was stirred at room temperature for 16 hours. The mixture was then acidified with hydrochloric acid and extracted with diethyl ether. The organic phase was dried and concentrated. The residue was stirred with diethyl ether and then filtered off with suction. This gave 0.24 g of 5-chloro-6-sec-butyl-7-trimethylsilylmethylamino[1,2,4]triazolo[1,5-a]pyrimidine (log p=3.34; HPLC content: 100%).

The compounds of the formula (I) listed in tables 1 to 5 below are or were also obtained analogously to the methods described above.

TABLE 1

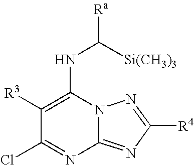

| Ex. | | | | |
|---|---|---|---|---|
| No. | R^{a} | R³ | R⁴ | logP |
| 1 | H | 2-Cl-4-F-phenyl | H | 3.61 |
| 2 | CH₃ | 2,4,6-trifluorophenyl | H | 3.71 |
| 3 | H | 2-Cl-6-F-phenyl | H | 3.43 |
| 4 | H | 2,4,6-trifluorophenyl | H | 3.39 |
| 5 | CH₃ | 2-Cl-6-F-phenyl | H | 3.79 |
| 6 | CH₃ | 2-Cl-4-F-phenyl | H | 3.95 |
| 7 | CH₃ | 2-Cl-phenyl | H | 3.83 |
| 8 | H | 3-Cl-5-(CF₃)-pyridin-2-yl | i-propyl | 4.51 |
| 9 | CH₃ | 5-F-pyrimidin-4-yl | i-propyl | 3.61 |
| 10 | CH₃ | 3-(CF₃)-pyridin-2-yl | H | 3.06 |
| 11 | CH₃ | 2-Cl-6-F-phenyl | Cyclopropyl | 4.65 |
| 12 | H | 2-Cl-6-F-phenyl | Cyclopropyl | 4.22 |
| 13 | H | 2,5-difluorophenyl | Cyclopropyl | 3.99 |
| 14 | CH₃ | 2,5-difluorophenyl | Cyclopropyl | 4.35 |
| 15 | CH₃ | 2,5-difluorophenyl | i-propyl | 4.58 |
| 16 | H | 2,5-difluorophenyl | i-propyl | 4.21 |
| 17 | CH₃ | 2,5-difluorophenyl | Methyl | 3.73 |
| 18 | H | 2,5-difluorophenyl | Methyl | 3.40 |
| 19 | CH₃ | 5-F-pyrimidin-4-yl | Cyclopropyl | 2.99 |
| 20 | H | 2-Cl-phenyl | Cyclopropyl | 4.23 |
| 21 | CH₃ | 2-Cl-phenyl | Cyclopropyl | 4.66 |
| 22 | H | 5-F-pyrimidin-4-yl | Cyclopropyl | 3.02 |
| 23 | CH₃ | 5-Cl-pyrimidin-4-yl | H | 2.70 |
| 24 | H | 5-Cl-pyrimidin-4-yl | H | 2.44 |
| 25 | CH₃ | sec-butyl | H | 3.87 |
| 26 | H | sec-butyl | H | 3.34 |
| 27 | H | 5-F-pyrimidin-4-yl | Methyl | 2.43 |
| 28 | CH₃ | 5-F-pyrimidin-4-yl | Methyl | 2.73 |
| 29 | H | N(—CH₂—CH₂—CH₂— | H | |
| | | CH₂—CH₂—) | | |
| 30 | CH₃ | N(—CH₂—CH₂—CH₂— | H | |
| | | CH₂—CH₂—) | | |
| 31 | H | N(—CHCH₃—CH₂—CH₂— | H | |
| | | CH₂—CH₂—) | | |
| 32 | H | N(—CHCH₃—CH₂—CH₂— | H | |
| | | CH₂—CH₂—) | | |
| 33 | H | N(—CHCH₃—CH₂—O—CH₂— | H | |
| | | CH₂—) | | |
| 34 | CH₃ | N(—CHCH₃—CH₂—O—CH₂— | H | |
| | | CH₂—) | | |
| 35 | H | 3,5-dimethylpyrazol-1-yl | H | |
| 36 | CH₃ | 3,5-dimethylpyrazol-1-yl | H | |
| 37 | H | N(CH₃)(C₂H₅) | H | |
| 38 | CH₃ | N(CH₃)(C₂H₅) | H | |
| 39 | H | 2,5-difluorophenyl | H | 3.26 |
| 40 | CH₃ | 2,5-difluorophenyl | H | 3.57 |
| 41 | H | 5-F-pyrimidin-4-yl | H | 2.28 |
| 42 | CH₃ | 5-F-pyrimidn-4-yl | H | 2.57 |

The logP values were determined in accordance with EEC Directive 79/831 Annex V. A8 by HPLC (gradient method, acetonitrile/0.1% aqueous phosphoric acid.

TABLE 2

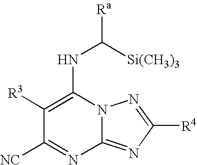

| Ex. | | | | |
|---|---|---|---|---|
| No. | R^{a} | R³ | R⁴ | logP |
| 43 | H | 2-Cl-4-F-phenyl | H | |
| 44 | CH₃ | 2,4,6-trifluorophenyl | H | |
| 45 | H | 2-Cl-6-F-phenyl | H | |
| 46 | H | 2,4,6-trifluorophenyl | H | |
| 47 | CH₃ | 2-Cl-6-F-phenyl | H | |
| 48 | CH₃ | 2-Cl-4-F-phenyl | H | |
| 49 | CH₃ | 2-Cl-phenyl | H | |
| 50 | H | 3-Cl-5-(CF₃)-pyridin-2-yl | i-propyl | |
| 51 | CH₃ | 5-F-pyrimidin-4-yl | i-propyl | |
| 52 | CH₃ | 3-(CF₃)-pyridin-2-yl | H | |
| 53 | CH₃ | 2-Cl-6-F-phenyl | Cyclopropyl | |
| 54 | H | 2-Cl-6-F-phenyl | Cyclopropyl | |
| 55 | H | 2,5-difluorophenyl | Cyclopropyl | |
| 56 | CH₃ | 2,5-difluorophenyl | Cyclopropyl | |
| 57 | CH₃ | 2,5-difluorophenyl | i-propyl | |
| 58 | H | 2,5-difluorophenyl | i-propyl | |
| 59 | CH₃ | 2,5-difluorophenyl | Methyl | |
| 60 | H | 2,5-difluorophenyl | Methyl | |
| 61 | CH₃ | 5-F-pyrimidin-4-yl | Cyclopropyl | |
| 62 | H | 2-Cl-phenyl | Cyclopropyl | |
| 63 | CH₃ | 2-Cl-phenyl | Cydopropyl | |
| 64 | H | 5-F-pyrimidin-4-yl | Cyclopropyl | |
| 65 | CH₃ | 5-Cl-pyrimidin-4-yl | H | |
| 66 | H | 5-Cl-pyrimidin-4-yl | H | |
| 67 | CH₃ | sec-butyl | H | |
| 68 | H | sec-butyl | H | |
| 69 | H | 5-F-pyrimidin-4-yl | Methyl | |
| 70 | CH₃ | 5-F-pyrimidin-4-yl | Methyl | |
| 71 | H | N(—CH₂—CH₂—CH₂— | H | |
| | | CH₂—CH₂—) | | |
| 72 | CH₃ | N(—CH₂—CH₂—CH₂— | H | |
| | | CH₂—CH₂—) | | |
| 73 | H | N(—CHCH₃—CH₂—CH₂— | H | |
| | | CH₂—CH₂—) | | |
| 74 | H | N(—CHCH₃—CH₂—CH₂— | H | |
| | | CH₂—CH₂—) | | |
| 75 | H | N(—CHCH₃—CH₂—O—CH₂— | H | |
| | | CH₂—) | | |
| 76 | CH₃ | N(—CHCH₃—CH₂—O— | H | |
| | | CH₂—CH₂—) | | |
| 77 | H | 3,5-dimethylpyrazol-1-yl | H | |
| 78 | CH₃ | 3,5-dimethylpyrazol-1-yl | H | |
| 79 | H | N(CH₃)(C₂H₅) | H | |
| 80 | CH₃ | N(CH₃)(C₂H₅) | H | |
| 81 | H | 2,5-difluorophenyl | H | |
| 82 | CH₃ | 2,5-difluorophenyl | H | |
| 83 | H | 5-F-pyrimidin-4-yl | H | |
| 84 | CH₃ | 5-F-pyrimidin-4-yl | H | |

The logP values were determined in accordance with EEC Directive 79/831 Annex V. A8 by HPLC (gradient method, acetonitrile/0.1% aqueous phosphoric acid.

TABLE 3

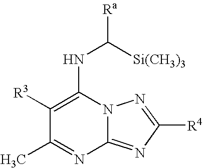

| Ex. | | | | |
|---|---|---|---|---|
| No. | R^{a} | R³ | R⁴ | logP |
| 85 | H | 2-Cl-4-F-phenyl | H | |
| 86 | CH₃ | 2,4,6-trifluorophenyl | H | |
| 87 | H | 2-Cl-6-F-phenyl | H | |
| 88 | H | 2,4,6-trifluorophenyl | H | |
| 89 | CH₃ | 2-Cl-6-F-phenyl | H | |
| 90 | CH₃ | 2-Cl-4-F-phenyl | H | |
| 91 | CH₃ | 2-Cl-phenyl | H | |
| 92 | H | 3-Cl-5-(CF₃)-pyridin-2-yl | i-propyl | |
| 93 | CH₃ | 5-F-pyrimidin-4-yl | i-propyl | |
| 94 | CH₃ | 3-(CF₃)-pyridin-2-yl | H | |
| 95 | CH₃ | 2-Cl-6-F-phenyl | Cyclopropyl | |
| 96 | H | 2-Cl-6-F-phenyl | Cyclopropyl | |
| 97 | H | 2,5-difluorophenyl | Cyclopropyl | |
| 98 | CH₃ | 2,5-difluorophenyl | Cyclopropyl | |
| 99 | CH₃ | 2,5-difluorophenyl | i-propyl | |
| 100 | H | 2,5-difluorophenyl | i-propyl | |
| 101 | CH₃ | 2,5-difluorophenyl | Methyl | |
| 102 | H | 2,5-difluorophenyl | Methyl | |
| 103 | CH₃ | 5-F-pyrimidin-4-yl | Cyclopropyl | |
| 104 | H | 2-Cl-phenyl | Cyclopropyl | |
| 105 | CH₃ | 2-Cl-phenyl | Cyclopropyl | |
| 106 | H | 5-F-pyrimidin-4-yl | Cyclopropyl | |
| 107 | CH₃ | 5-Cl-pyrimidin-4-yl | H | |
| 108 | H | 5-Cl-pyrimidin-4-yl | H | |
| 109 | CH₃ | sec-butyl | H | |
| 110 | H | sec-butyl | H | |
| 111 | H | 5-F-pyrirnidin-4-yl | Methyl | |
| 112 | CH₃ | 5-F-pyrimidin-4-yl | Methyl | |
| 113 | H | N(—CH₂—CH₂—CH₂— | H | |
| | | CH₂—CH₂—) | | |
| 114 | CH₃ | N(—CH₂—CH₂—CH₂—CH₂— | H | |
| | | CH₂—) | | |
| 115 | H | N(—CHCH₃—CH₂—CH₂—CH₂— | H | |
| | | CH₂—) | | |
| 116 | H | N(—CHCH₃—CH₂—CH₂—CH₂— | H | |
| | | CH₂—) | | |
| 117 | H | N(—CHCH₃—CH₂—O—CH₂— | H | |
| | | CH₂—) | | |
| 118 | CH₃ | N(—CHCH₃—CH₂—O—CH₂— | H | |
| | | CH₂—) | | |
| 119 | H | 3,5-dimethylpyrazol-1-yl | H | |
| 120 | CH₃ | 3,5-dimethylpyrazol-1-yl | H | |
| 121 | H | N(CH₃)(C₂H₅) | H | |
| 122 | CH₃ | N(CH₃)(C₂H₅) | H | |
| 123 | H | 2,5-difluorophenyl | H | |
| 124 | CH₃ | 2,5-difluorophenyl | H | |
| 125 | H | 5-F-pyrimidin-4-yl | H | |
| 126 | CH₃ | 5-F-pyrimidin-4-yl | H | |

The logP values were determined in accordance with EEC Directive 79/831 Annex V. A8 by HPLC (gradient method, acetonitrile/0.1% aqueous phosphoric acid.

TABLE 4

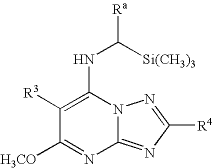

| Ex. | | | | |
|---|---|---|---|---|
| No. | R^{a} | R³ | R⁴ | logP |
| 127 | H | 2-Cl-4-F-phenyl | H | |
| 128 | CH₃ | 2,4,6-trifluorophenyl | H | |
| 129 | H | 2-Cl-6-F-phenyl | H | |
| 130 | H | 2,4,6-trifluorophenyl | H | |
| 131 | CH₃ | 2-Cl-6-F-phenyl | H | |
| 132 | CH₃ | 2-Cl-4-F-phenyl | H | |
| 133 | CH₃ | 2-Cl-phenyl | H | |
| 134 | H | 3-Cl-5-(CF₃)-pyridin-2-yl | i-propyl | |
| 135 | CH₃ | 5-F-pyrimidin-4-yl | i-propyl | |
| 136 | CH₃ | 3-(CF₃)-pyridin-2-yl | H | |
| 137 | CH₃ | 2-Cl-6-F-phenyl | Cyclopropyl | |
| 138 | H | 2-Cl-6-F-phenyl | Cyclopropyl | |
| 139 | H | 2,5-difluorophenyl | Cyclopropyl | |
| 140 | CH₃ | 2,5-difluorophenyl | Cyclopropyl | |
| 141 | CH₃ | 2,5-difluorophenyl | i-Propyl | |
| 142 | H | 2,5-difluorophenyl | i-Propyl | |
| 143 | CH₃ | 2,5-difluorophenyl | Methyl | |
| 144 | H | 2,5-difluorophenyl | Methyl | |
| 145 | CH₃ | 5-F-pyrimidin-4-yl | Cyclopropyl | |
| 146 | H | 2-Cl-phenyl | Cyclopropyl | |
| 147 | CH₃ | 2-Cl-phenyl | Cyclopropyl | |
| 148 | H | 5-F-pyrimidin-4-yl | Cyclopropyl | |
| 149 | CH₃ | 5-Cl-pyrimidin-4-yl | H | |
| 150 | H | 5-Cl-pyrimidin-4-yl | H | |
| 151 | CH₃ | sec-butyl | H | |
| 152 | H | sec-butyl | H | |
| 153 | H | 5-F-pyrimidin-4-yl | | |
| 154 | CH₃ | 5-F-pyrimidin-4-yl | | |
| 155 | H | N(—CH₂—CH₂—CH₂—CH₂— | H | |
| | | CH₂—) | | |
| 156 | CH₃ | N(—CH₂—CH₂—CH₂—CH₂— | H | |
| | | CH₂—) | | |
| 157 | H | N(—CHCH₃—CH₂—CH₂—CH₂— | H | |
| | | CH₂—) | | |
| 158 | H | N(—CHCH₃—CH₂—CH₂—CH₂— | H | |
| | | CH₂—) | | |
| 159 | H | N(—CHCH₃—CH₂—O—CH₂— | H | |
| | | CH₂—) | | |
| 160 | CH₃ | N(—CHCH₂—CH₂—O—CH₂— | H | |
| | | CH₂—) | | |
| 161 | H | 3,5-dimethylpyrazol-1-yl | H | |
| 162 | CH₃ | 3,5-dimethylpyrazol-1-yl | H | |
| 163 | H | N(CH₃)(C₂H₅) | H | |
| 164 | CH₃ | N(CH₃)(C₂H₅) | H | |
| 165 | H | 2,5-difluorophenyl | H | |
| 166 | CH₃ | 2,5-difluorophenyl | H | |
| 167 | H | 5-F-pyrimidin-4-yl | H | |
| 168 | CH₃ | 5-F-pyrimidin-4-yl | H | |

The logP values were determined in accordance with EEC Directive 79/831 Annex V. A8 by HPLC (gradient method: acetonitrile/0.1% aqueous phosphoric acid).

TABLE 5

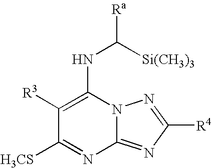

| Ex. | | | | |
|---|---|---|---|---|
| No. | R^{a} | R³ | R⁴ | logP |
| 169 | H | 2-Cl-4-F-phenyl | H | |
| 170 | CH₃ | 2,4,6-trifluorophenyl | H | |
| 171 | H | 2-Cl-6-F-phenyl | H | |
| 172 | H | 2,4,6-trifluorophenyl | H | |
| 173 | CH₃ | 2-Cl-6-F-phenyl | H | |
| 174 | CH₃ | 2-Cl-4-F-phenyl | H | |
| 175 | CH₃ | 2-Cl-phenyl | H | |
| 176 | H | 3-Cl-5-(CF₃)-pyridin-2-yl | i-propyl | |
| 177 | CH₃ | 5-F-pyrimidin-4-yl | i-propyl | |
| 178 | CH₃ | 3-(CF₃)-pyridin-2-yl | H | |
| 179 | CH₃ | 2-Cl-6-F-phenyl | Cyclopropyl | |
| 180 | H | 2-Cl-6-F-phenyl | Cyclopropyl | |
| 181 | H | 2,5-difluorophenyl | Cyclopropyl | |
| 182 | CH₃ | 2,5-difluorophenyl | Cyclopropyl | |
| 183 | CH₃ | 2,5-difluorophenyl | i-propyl | |
| 184 | H | 2,5-difluorophenyl | i-propyl | |
| 185 | CH₃ | 2,5-difluorophenyl | Methyl | |
| 186 | H | 2,5-difluorophenyl | Methyl | |
| 187 | CH₃ | 5-F-pyrimidin-4-yl | Cyclopropyl | |
| 188 | H | 2-Cl-phenyl | Cyclopropyl | |
| 189 | CH₃ | 2-Cl-phenyl | Cyclopropyl | |
| 190 | H | 5-F-pyrimidin-4-yl | Cyclopropyl | |
| 191 | CH₃ | 5-Cl-pyrimidin-4-yl | H | |
| 192 | H | 5-Cl-pyrimidin-4-yl | H | |
| 193 | CH₃ | sec-butyl | H | |
| 194 | H | sec-butyl | H | |
| 195 | H | 5-F-pyrimidin-4-yl | Methyl | |
| 196 | CH₃ | 5-F-pyrimidin-4-yl | Methyl | |
| 197 | H | N(—CH₂—CH₂—CH₂—CH₂— | H | |
| | | CH₂—) | | |
| 198 | CH₃ | N(—CH₂—CH₂—CH₂—CH₂— | H | |
| | | CH₂—) | | |
| 199 | H | N(—CHCH₃—CH₂—CH₂—CH₂— | H | |
| | | CH₂—) | | |
| 200 | H | N(—CHCH₃—CH₂—CH₂—CH₂— | H | |
| | | CH₂—) | | |
| 201 | CH₃ | N(—CHCH₃—CH₂—O—CH₂— | H | |
| | | CH₂—) | | |
| 202 | CH₃ | N(—CHCH₃—CH₂—O—CH₂— | H | |
| | | CH₂—) | | |
| 203 | H | 3,5-dimethylpyrazol-1-yl | H | |
| 204 | CH₃ | 3,5-dimethylpyrazol-1-yl | H | |
| 205 | H | N(CH₃)(C₂H₅) | H | |
| 206 | CH₃ | N(CH₃)(C₂H₅) | H | |
| 207 | H | 2,5-difluorophenyl | H | |
| 208 | CH₃ | 2,5-difluorophenyl | H | |
| 209 | H | 5-F-pyrimidin-4-yl | H | |
| 210 | CH₃ | 5-F-pyrimidin-4-yl | H | |

The logP values were determined in accordance with EEC Directive 79/831 Annex V. A8 by HPLC (gradient method: acetonitrile/0.1% aqueous phosphoric acid).

### USE EXAMPLES

### Example A

Podosphaera-test (Apple)/protective

| | |
|---|---|
| Solvents: | 24.5 parts by weight of acetone |
| | 24.5 parts by weight of dimethylacetamide |
| Emulsifier: | 1 part by weight of alkyl aryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the apple mildew pathogen Podosphaera leucotricha. The plants are then placed in a greenhouse at about 23° C. and a relative atmospheric humidity of about 70%.

Evaluation is carried out 10 days after the inoculation. 0% means an efficacy which corresponds to that of the control, whereas an efficacy of 100% means that no infection is observed.

In this test, the compounds according to the invention of examples 1, 2, 3, 4, 5 and 6 showed, at an application rate of 100 g/ha, an efficacy of more than 90%.

### Example B

Venturia - Test (Apple)/protective

| | |
|---|---|
| Solvents: | 24.5 parts by weight of acetone |
| | 24.5 parts by weight of dimethylacetamide |
| Emulsifier: | 1 part by weight of alkyl aryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compounds according to the invention of examples 1, 2, 3, 4, 5 and 6 showed, at in application rate of 100 g/ha, an efficacy of more than 90%.

### Example C

Botrytis - Test (Bean)/protective

| | |
|---|---|
| Solvents: | 24.5 parts by weight of acetone |
| | 24.5 parts by weight of dimethylacetamide |
| Emulsifier: | 1 part by weight of alkyl aryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, 2 small pieces of agar colonised by Botrytis cinerea are placed onto each leaf. The inoculated plants are placed in a dark chamber at about 20° C. and 100% relative atmospheric humidity.

The size of the infected areas on the leaves is evaluated 2 days after the inoculation. 0% means an efficacy which corresponds to that of the control, whereas an efficacy of 100% means that no infection is observed.

In this test, the compounds according to the invention of examples 1, 2, 4 and 6 showed, at an application rate of 500 g/ha, an efficacy of more than 90%.

### Example D

Sphaerotheca-Test (Cucumber)/protective

| | | |
|---|---|---|
| Solvent: | 49 | parts by weight of N,N-dimethylformamide |
| Emulsifier: | 1 | part by weight of alkylaryl polyglycol ether |

To produce a suitable preparation of active compound, 1 part by weight of active compound is mixed with the stated amounts of solvent and emulsifier, then the concentrate is diluted with water to the desired concentration.

To test for protective activity, young cucumber plants are sprayed with the preparation of active compound at the stated application rate. 1 day after the treatment, the plants are inoculated with a spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

In this test, the compounds according to the invention of examples 4, 15, 17 and 24 showed, at an application rate of 750 g/ha, an efficacy of more than 90%.

1-10. (canceled)

11: A triazolopyrimidine of formula (i) 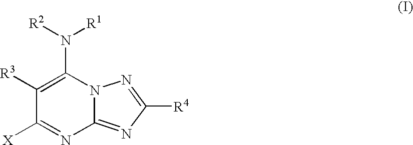
in which
R¹ represents H, optionally substituted alkyl, optionally substituted alkenyl, optionally substituted alkynyl, optionally substituted cyloalkyl, or optionally substituted heterocyclyl; or represents an organic radical that contains 3 to 13 carbon atoms and one or more silicon atoms and, optionally, 1 to 3 identical or different heteroatoms selected from the group consisting of oxygen, nitrogen, and sulfur, and that is unsubstituted or substituted by 1 to 4 identical or different halogens;
R² represents an organic radical that contains 3 to 13 carbon atoms and one or more silicon atoms and, optionally, 1 to 3 identical or different heteroatoms selected from the group consisting of oxygen, nitrogen, and sulfur, and that is unsubstituted or substituted by 1 to 4 identical or different halogens, or
R¹ and R² together with the nitrogen atom to which they are attached represent an optionally substituted heterocyclic ring that contains one or more silicon atoms and/or is substituted by one or more radicals R²,
R³ represents optionally substituted aryl, optionally substituted heterocyclyl, optionally substituted alkyl, optionally substituted alkenyl, optionally substituted alkynyl, optionally substituted cycloalkyl optionally substituted aralkyl, optionally substituted amino, optionally substituted (C₁-C₈)-alkoxy, optionally substituted (C₁-C₈)-alkylthio, optionally substituted (C₆-C₁₀)-aryloxy, optionally substituted (C₆-C₁₀)-arylthio, optionally substituted heterocyclyloxy, optionally substituted heterocyclyloxy, optionally substituted C₆-C₁₀)-aryl-(C₁-C₄)-alkoxy, optionally substituted (C₆-C₁₀)-aryl-(C₁-C₄)-alkylthio, optionally substituted heterocyclyl-(C₁-C₄)-alkoxy, or optionally substituted heterocyclyl-(C₁-C₄)-alkylthio;
R⁴ represents H, halogen, optionally halogen-substituted alkyl, or optionally halogen-substituted cycloalkyl, and
X represents halogen, cyano, optionally substituted alkyl, optionally substituted alkoxy, or optionally substituted phenyl.

12: A triazolopyrimidine of formula (I) as claimed in claim 11 where
R¹ represents H; represents alkyl having 1 to 6 carbon atoms that is optionally mono- to pentasubstituted by identical or different substituents selected from the group consisting of halogen, cyano, hydroxy, alkoxy having 1 to 4 carbon atoms, and cycloalkyl having 3 to 8 carbon atoms; represents alkenyl having 2 to 6 carbon atoms that is optionally mono- to trisubstituted by identical or different substituents selected from the group consisting of halogen, cyano, hydroxy, alkoxy having 1 to 4 carbon atoms, and cycloalkyl having 3 to 8 carbon atoms; represents alkynyl having 3 to 6 carbon atoms that is optionally mono- to trisubstituted by identical or different substituents selected from the group consisting of halogen, cyano, alkoxy having 1 to 4 carbon atoms, and cycloalkyl having 3 to 8 carbon atoms; represents cycloalkyl having 3 to 8 carbon atoms that is optionally mono- to trisubstituted by identical or different substituents selected from the group consisting of halogen and alkyl having 1 to 4 carbon atoms; represents saturated or unsaturated heterocyclyl having 3 to 8 ring members and 1 to 3 heteroatoms selected from the group consisting of nitrogen, oxygen, and sulfur, where the heterocyclyl is optionally mono- or disubstituted by halogen, alkyl having 1 to 4 carbon atoms, cyano, and/or cycloalkyl having 3 to 8 carbon atoms; or represents an aliphatic saturated or unsaturated group having 1 to 13 carbon atoms and one or more silicon atoms that optionally contains 1 to 3 identical or different heteroatoms selected from the group consisting of oxygen, sulfur, and nitrogen and that is unsubstituted or substituted by 1 to 4 identical or different halogen atoms,
R² represents an aliphatic saturated or unsaturated group having 1 to 13 carbon atoms and one or more silicon atoms that optionally contains 1 to 3 identical or different heteroatoms selected from the group consisting of oxygen, sulfur, and nitrogen and which is unsubstituted or substituted by 1 to 4 identical or different halogen atoms, or
R¹ and R² together with the nitrogen atom to which they are attached represent a saturated or unsaturated heterocyclic ring having 3 to 8 ring members that contains one or more silicon atoms and/or is substituted by one or more radicals R², where the heterocycle optionally contains a further nitrogen, oxygen, or sulfur atom as ring member and where the heterocycle is optionally substituted up to three times by fluorine, chlorine, bromine, alkyl having 1 to 4 carbon atoms, and/or haloalkyl having 1 to 4 carbon atoms and 1 to 9 fluorine and/or chlorine atoms,
R³ represents C₁-C₁₀-alkyl, C₂-C₁₀-alkenyl, C₂-C₁₀-alkynyl, C₃-C₈-cycloalkyl, or phenyl-C₁-C₁₀-alkyl, where each such group is unsubstituted or partly or fully halogenated and/or optionally carries one to three radicals R^{x}; represents C₁-C₁₀-halogenalkyl that optionally carries one to three radicals R^{x}; represents phenyl that is optionally mono- to tetrasubstituted by identical or different substituents selected from the group consisting of halogen, cyano, nitro, amino, hydroxy, formyl, carboxy, carbamoyl, and thiocarbamoyl, of straight-chain or branched alkyl, alkoxy, alkylthio, alkylsulfinyl, and alkylsulfonyl having in each case 1 to 6 carbon atoms, of straight-chain or branched alkenyl and alkenyloxy having in each case 2 to 6 carbon atoms, of straight-chain or branched haloalkyl, haloalkoxy, haloalkylthio, haloalkylsulfinyl, and haloalkylsulfonyl having in each case 1 to 6 carbon atoms and 1 to 13 identical or different halogen atoms, of straight-chain or branched haloalkenyl and haloalkenyloxy having in each case 2 to 6 carbon atoms and 1 to 11 identical or different halogen atoms, of straight-chain or branched alkylamino, dialkylamino, alkylcarbonyl, alkylcarbonyloxy, alkoxycarbonyl, alkylsulfonyloxy, hydroximinoalkyl, and alkoximinoalkyl having in each case 1 to 6 carbon atoms in the individual alkyl moieties, of cycloalkyl having 3 to 8 carbon atoms, and of 2,3-attached 1,3-propanediyl, 1,4-butanediyl, methylenedioxy (—O—CH₂—O—), and 1,2-ethylenedioxy (—O—CH₂—CH₂—O—), each of which is optionally mono- or polysubstituted by identical or different substituents selected from the group consisting of halogen, alkyl having 1 to 4 carbon atoms, and haloalkyl having 1 to 4 carbon atoms and 1 to 9 identical or different halogen atoms; represents saturated or unsaturated heterocyclyl having 3 to 8 ring members and 1 to 3 heteroatoms selected from the group consisting of nitrogen, oxygen, and sulfur, where the heterocyclyl is optionally mono- or disubstituted by halogen, alkyl having 1 to 4 carbon atoms, alkoxy having 1 to 4 carbon atoms, alkylthio having 1 to 4 carbon atoms, haloalkoxy having 1 to 4 carbon atoms, haloalkylthio having 1 to 4 carbon atoms, cyano, nitro, and/or cycloalkyl having 3 to 6 carbon atoms; or represents C₁-C₈-alkylamino, C₂-C₈-alkenylamino, C₂-C₈-alkynylamino, di-C₁-C₈-alkylamino, di-C₂-C₈-alkenylamino, di-C₂-C₈-alkynylamino, C₂-C₈-alkenyl-(C₂-C₈)-alkynylamino, C₂-C₆-alkynyl-(C₁-C₈)-alkylamino, C₂-C₈-alkenyl-(C₁-C₈)-alkylamino, C₆-C₁₀-arylamino, C₆-C₁₀-aryl-(C₁-C₈)-alkylamino, C₆-C₁₀-aryl-(C₁-C₄)-alkyl-(C₁-C₈)-alkylamino, heterocyclyl-(C₁-C₈)-alkylamino, or heterocyclyl-(C₁-C₄)-alkyl-(C₁-C₈)-alkylamino;
where R^{x} represents cyano, nitro, hydroxy, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-halogenalkylthio, C₁-C₆-alkylsulfinyl, C₁-C₆-halogenalkylsulfinyl, C₁-C₆-alkylsulfonyl, C₁-C₆-halogenalkylsulfonyl, C₁-C₆-alkylamino, di-C₁-C₆-alkylamino, C₂-C₆-alkenyl, C₂-C₆-alkenyloxy, C₂-C₆-alkynyl, or C₃-C₆-alkynyloxy, or represents optionally halogenated oxy-C₁-C₄-alkyl-C₁-C₄-alkeneoxy, oxy-C₁-C₄-alkenyl-C₁-C₄-alkoxy, or oxy-C₁-C₄-alkyl-C₁-C₄-alkyloxy,
R⁴ represents H, halogen, (C₁-C₄)-alkyl that is unsubstituted or substituted by one or more halogen atoms, or cyclopropyl that is unsubstituted or substituted by one or more halogen atoms, and
X represents fluorine, chlorine, bromine, CN, (C₁-C₄)-alkyl that is unsubstituted or substituted by one or more fluorine or chlorine atoms, (C₁-C₄)-alkoxy that is unsubstituted or substituted by one or more fluorine or chlorine atoms, or (C₁-C₄)-alkylthio that is unsubstituted or substituted by one or more fluorine or chlorine atoms.

13: A triazolopyrimidine of formula (I) as claimed in claim 11 where
R¹ represents hydrogen, methyl, or ethyl,
R² represents a group of the formula Y²—Si(OₘCH₃)(OₙCH₃)(OₚY³), where
m, n, and p independently of one another represent 0 or 1;
Y² represents a bond or alkanediyl, alkenediyl, or alkynediyl, each of which is straight-chain or branched, has 1 to 6 or 2 to 6 carbon atoms, is optionally interrupted by one or two nonadjacent oxygen atoms, and is unsubstituted or substituted by one to three identical or different halogen atoms; and
Y³ represents straight-chain or branched alkyl or alkenyl having 1 to 5 or 2 to 5 carbon atoms, optionally interrupted by an oxygen-nitrogen or sulfur atom and unsubstituted or substituted by 1 to 3 identical or different halogen atoms;
R³ represents (C₁-C₈)-alkyl, (C₁-C₈)-cycloalkyl, or benzyl; represents phenyl that is optionally mono- to trisubstituted by identical or different substituents selected from the group consisting of fluorine, chlorine, bromine, cyano, nitro, formyl, methyl, ethyl, n- or i-propyl, n-, i-, s-, or t-butyl, allyl, propargyl, methoxy, ethoxy, n- or i-propoxy, methylthio, ethylthio, n- or i-propylthio, methylsulfinyl, ethylsulfinyl, methylsulfonyl, ethylsulfonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulfinyl, trifluoromethylsulfonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl, and cyclohexyl, and of 2,3-attached 1,3-propanediyl, 1,4-butanediyl, methylenedioxy (—O—CH₂—O—), and 1,2-ethylenedioxy (—O—CH₂—CH₂—O—), each of which is optionally mono- or polysubstituted by identical or different substituents selected from the group consisting of fluorine, chlorine, methyl, ethyl, n-propyl, i-propyl, and trifluoromethyl; represents pyridyl that is attached in the 2- or 4-position and is optionally mono- to tetrasubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl;
represents pyrimidyl that is attached in the 2- or 4-position and is optionally mono- to trisubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl; represents thienyl that is attached in the 2- or 3-position and is optionally mono- to trisubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl; represents C₁-C₈-alkylamino or di-C₁-C₈-alkylamino;
represents thiazolyl that is attached in the 2-, 4- or 5-position and is optionally mono- to trisubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl; or represents N-piperidinyl, N-tetrazolyl, N-pyrazolyl, N-imidazolyl, N-1,2,4-triazolyl, N-pyrrolyl, or N-morpholinyl, each of which is unsubstituted or mono- or polysubstituted by identical or different substituents selected from the group consisting of fluorine, chlorine, bromine, cyano, nitro, methyl, ethyl, methoxy, methylthio, hydroximinomethyl, hydroximinoethyl, methoximinomethyl, methoximinoethyl, and trifluoromethyl,
R⁴ represents H, Cl, F, CH₃, —CH(CH₃)₂, or cyclopropyl; and
X represents F, Cl, CN, (C₁-C₄)-alkyl that is unsubstituted or substituted by one or more fluorine or chlorine atoms, OCH₃, or SCH₃.

14: A triazolopyrimidine of formula (I) as claimed in claim 11, where
R¹ represents H;
R² represents SiMe₃, SiMe₂Et, SiMe₂CHMe₂, SiMe₂CH₂CHMe₂, SiMe₂CH₂CMe₃, SiMe₂OCHMe₂, SiMe₂OCH₂CHMe₂, CH₂SiMe₃, CH₂SiMe₂Et, CH₂SiMe₂CHMe₂, CH₂SiMe₂CH₂CHMe, CH₂SiMe₂OMe, CH₂SiMe₂OCHMe₂, CH₂SiMe₂OCH₂CHMe₂, CHMeSiMe₃, CHMeSiMe₂OMe, (CH₂)₂SiMe₃, (CH₂)₂SiMe₂Et, (CH₂)₂SiMe₂CHMe₂, (CH₂)₂SiMe₂CMe₃, (CH₂)₂SiMe₂CH₂CHMe₂, (CH₂)₂SiMe₂CH₂CH₂Me, (CH₂)₂SiMe₂CH₂CMe₃, (CH₂)₂SiMe₂OCHMe₂, (CH₂)₂SiMe₂OCH₂CHMe₂, CHMeCH₂SiMe₃, CHMeCH₂SiMe₂Et, CHMeCH₂SiMe₂CH₂CH₂Me, CHMeCH₂SiMe₂CHMe₂, CHMeCH₂SiMe₂CMe₃, CHMeCH₂SiMe₂CH₂CHMe₂, CFMeCH₂SiMe₃, CHMeCH₂CH₂SiMe₂OMe, CHMeCH₂SiMe₂OCHMe₂, CHMeCH₂SiMe₂OCH₂CHMe₂, CH₂CHMeSiMe₃, CH₂CHMeSiMe₂Et, CH₂CHMeSiMe₂CHMe₂, CHMeCHMeSiMe₃, CMe₂CH₂SiMe₃, (CH₂)₃SiMe₃, (CH₂)₃SiMe₂Et, (CH₂)₃SiMe₂CHMe₂, (CH₂)₃SiMe₂CH₂CHMe₂, (CH₂)₃SiMe₂OMe, (CH₂)₃SiMe₂OCHMe₂, (CH₂)₃SiMe₂OCH₂CHMe₂, CHMeCH₂CH₂SiMe₃, CHMeCH₂CH₂SiMe₂Et, CHMeCH₂CH₂SiMe₂CHMe₂, CHMeCH₂CH₂CH₂SiMe₂OMe, CHMeCH₂CH₂SiMe₂OCHMe₂, CMe=CHSiMe₃, CH₂CH₂SiMe₂OMe, —C≡C—SiMe₃, —CH₂—C≡C—SiMe₃, or —CHMe-C≡C—SiMe₃;
R³ represents (C₁-C₆)-alkyl, (C₃₋₆)-alkenyl, (C₃-C₆)-alkynyl, or (C₃-C₈)-cycloalkyl, where each such group is unsubstituted or substituted by one or more fluorine or chlorine atoms; represents 2,4- or 2,6-disubstituted phenyl, 2-substituted phenyl, or 2,4,6-trisubstituted phenyl; represents pyridyl that is attached in the 2- or 4-position and that is optionally mono- to tetrasubstituted by identical or different substituents selected from the group consisting of fluorine, chlorine, bromine, cyano, methyl, ethyl, methoxy, methylthio, hydroxyiminomethyl, hydroximinoethyl, methoximinomethyl, methoximinoethyl, and trifluoromethyl; or represents pyrimidyl that is attached in the 4-position and that is optionally mono- to trisubstituted by identical or different substituents selected from the group consisting of fluorine, chlorine, bromine, cyano, methyl, ethyl, methoxy, methylthio, hydroximinomethyl, hydroximinoethyl, methoximinomethyl, methoximinoethyl, and trifluoromethyl;
R⁴ represents H, —CH₃, —CH(CH₃)₂, Cl, or cyclopropyl, and
X represents fluorine, chlorine, CN, (C₁-C₃)-alkyl, or (C₁-C₃)-haloalkyl, OCH₃, or SCH₃.

15: A process for preparing a triazolopyrimidine of formula (I) as claimed in claim 11 comprising reacting a halotriazolopyrimidine of formula (II) 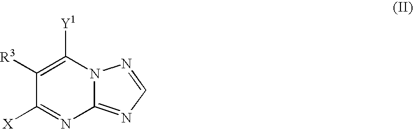
in which
R³ and X are as defined for formula (I) in claim 11, and
Y¹ represents halogen,
with an amine of formula (III) 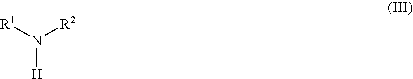
in which R¹ and R² are as defined for formula (I) in claim 11, optionally in the presence of a diluent, optionally in the presence of an acid acceptor, and optionally in the presence of a catalyst.

16: A composition for controlling unwanted microorganisms comprising one or more triazolopyrimidines of formula (I) as claimed in claim 11 and one or more extenders and/or surfactants.

17: A composition as claimed in claim 16 additionally comprising one or more additional fungicidally or insecticidally active compound.

18: A method for controlling unwanted microorganisms comprising applying an effective amount of a triazolopyrimidine of formula (I) as claimed in claim 11 to the unwanted microorganisms and/or their habitat.

19: A method for preparing compositions for controlling unwanted microorganisms comprising mixing one or more triazolopyrimidines of formula (I) as claimed in claim 11 with one or more extenders and/or surfactants.
